(19) United States
(12) Patent Application Publication
(10) Pub. No.: US 2018/0104486 A1
(43) Pub. Date: Apr. 19, 2018

(54) APPARATUS AND METHOD FOR TREATING CANCER USING DISCONTINUOUS FRACTIONAL ALTERNATIVE ELECTRIC FIELD

(71) Applicants: Myong Geun Yoon, Gyeonggi-do (KR); Korea University Research and Business Foundation, Seoul (KR)

(72) Inventors: Myong Geun Yoon, Gyeonggi-do (KR); Jiwon Sung, Gyeonggi-do (KR); Yunhui Jo, Seoul (KR); Eun Ho Kim, Seoul (KR)

(21) Appl. No.: 15/296,864

(22) Filed: Oct. 18, 2016

(51) Int. Cl. A61N 1/36; A61N 1/04

(52) U.S. Cl. CPC A61N 1/36014; A61N 1/0476

(57) ABSTRACT

The present invention relates to an apparatus and a method for treating cancer using a discontinuous fractional alternative electric field, and more particularly, to an apparatus and a method for treating cancer using a discontinuous fractional alternative electric field capable of providing the same treatment effect on tumor while minimizing an effect on normal tissues by reducing time for which an electric field is applied over the existing electric field treatment. According to the exemplary embodiments of the present invention, the discontinuous fractional alternative electric field shows the same effect as applying the continuous electric field to the cancer cells while reducing the effect on the normal cells, and as a result the method for treating cancer using an optimized alternative electric field may be constructed by the treatment using the discontinuous fractional alternative electric field.

### TECHNICAL FIELD

The present invention relates to an apparatus and a method for treating cancer using a discontinuous fractional alternative electric field, and more particularly, to an apparatus and a method for treating cancer using a discontinuous fractional alternative electric field capable of providing the same treatment effect on tumor while minimizing an effect on normal tissues by reducing time for which an electric field is applied over the existing electric field treatment.

### BACKGROUND

A cell division is made by combining microtubules having a dipole moment with each other by an electric force when the microtubules are divided to generate a spindle and allowing the generated spindle to pull chromosomes arrayed at a center of cells to both sides. If the division of cancer cells is delayed, the cancer may be treated by the suppression of the division of the cancer cells. Recently, researches on treating cancer by suppressing a division of cancer cells using an alternative electric field have been conducting.

The movement of the microtubules for an electric field applied to the outside of cells to form a spindle may be controlled and cells in which the spindle is not properly formed by the electric field are not divided normally. However, researches have been mostly focused on only an effect of alternative electric field on cancer cells and researches on an effect of electric field on normal cells have rarely been done. These researches assert that since the division of normal cells has a longer division time than the division of cancer cells, the effect of electric field on the normal cells is insignificant. However, since polar substances affected by the electric field are present even in a human body as well as the normal cells, there is a possibility that the polar substances are affected by the electric field, which is not yet clearly found.

Further, since the electric field treatment for glioblastoma that has been currently approved by FDA and has been actually performed needs to be continuously performed over 18 hours a day while an electric pad applying an electric field is attached to a skin, side effects such as headache, displeasure, muscle cramps, and skirt ulcer that are caused by the exposure to the electric field over a long time have been reported. Therefore, a technology of causing less side effects on a human tissue while showing a treatment effect like treating tumor using a continuous electric field is required.

To reduce an effect of radiation applied to normal tissues other than a treatment target, even radiotherapy has performed discontinuous fractional irradiation. Further, a method for treating disease by changing a flow of ion using a continuous or discontinuous induction current is disclosed in KR 10-2005-0045995, but treatment using an alternative electric field has not yet been known. U.S. Pat. No. 8,718,756 B2 proposed a method for applying an electric field using different pairs of electrodes in different directions, not the existing method for using a pair of electrodes as a method for improving an effect on multiplying cells, that is, cancer cells by optimizing a condition of electric field, which is like applying a continuous electric field whose only direction is changed to cells.

Therefore, the present inventors have tried to find out a treatment method for less affecting normal tissues while showing the same cancer treatment effect as the existing treatment using an electric field. As a result, the present inventors completed the present invention by confirming that the cancer treatment using a discontinuous fractional alternative electric field has the same effect as applying a continuous electric field to tumor while minimizing an effect on normal tissues.

### SUMMARY

An object of the present invention is to provide a method and an apparatus for treating cancer using an optimized discontinuous fractional electric field showing the same treatment effect as applying a continuous electric field to tumor while reducing an effect of alternative electric field on normal tissues.

To achieve the above object, the present invention provides an apparatus for suppressing a division of cancer cells or inducing a apoptosis of cancer cells by a system for generating a discontinuous fractional alternative electric field including: an alternative electric field generator; an alternative electric field controller; an alternative electric field amplifier; and an alternative electric field transmitter, wherein the alternative electric field generator generates an alternative voltage of 100 to 300 kHz and 1 to 20 Vpp, the controller sets an on/off time and period of the electric field to generate a discontinuous fractional alternative voltage, and the alternative electric field amplifier amplifies a voltage transmitted from the controller up to 800 Vpp, and the transmitter transmits the amplified voltage through.

The present invention also provides a method for treating cancer using the apparatus of above, the method comprises: a) generating an alternative voltage of 100 to 300 kHz and 1 to 20 Vpp in the alternative electric field generator; b) setting an on/off time and period of the electric field thereby generating a discontinuous fractional alternative voltage; c) amplifying the discontinuous fractional alternative voltage generated in step b) up to 800 Vpp in the alternative electric field amplifier; and d) transmitting the generated voltage to a treatment site using an electric pad.

### BRIEF DESCRIPTION OF THE DRAWINGS

FIG. 1 is a graph illustrating a difference in time for which an electric field is actually applied according to methods for treating cancer using a continuous electric field and a discontinuous electric field for a total of treatment period.

FIG. 2 is a schematic diagram of a system for treating cancer using a discontinuous fractional alternative electric field.

FIG. 3 is a diagram illustrating a result of suppressing a division of cancer cells when an electric field is repeatedly applied for three days by making an on time of electric field a day different.

FIG. 4 is a diagram illustrating a comparison result of electric field effects when an on and off time of alternative electric field is different but a ratio of alternative electric field is 1:1.

FIG. 5 is a diagram illustrating a comparison result of effects when an alternative electric field having a magnitude of 0.9 and 1.2 v/cm is applied to cancer cells and normal cells, respectively by making an on time of electric field a day different.

FIG. 6 is a diagram illustrating a result of confirming how much a discontinuous fractional alternative electric field affects cancer cells and normal cells through biological factors when the electric field is applied to the cancer cells and the normal cells.

### DETAILED DESCRIPTION OF EMBODIMENTS

Unless differently defined, all the technical and scientific terms used in the present specification have the same meaning as understood by those skilled in the art to which the present invention pertains. Generally, the nomenclatures used in the present specification are well known in the present technical field and have been generally used.

The present invention generates a digital signal every constant period by using a program controlling an alternative electric field function generator, uses the generated digital signal as a trigger, and then generates a clock as much as desired time every time the trigger is generated and generates an electric field while the clock is generated. The electric field generated from the alternative electric field function generator is amplified by an amplifier to acquire a high voltage and then the acquired high voltage is applied between insulating electric wires, such that cells may be affected by a predetermined amount of electric field.

The present invention may periodically change an on/off time of electric field during treatment, a frequency, and a voltage. Further, the present invention may be applied to all cancers regardless of a kind of cancers.

Therefore, an aspect of the present invention relates to an apparatus for suppressing a division of cancer cells or inducing a apoptosis of cancer cells by a system for generating a discontinuous fractional alternative electric field including: an alternative electric field generator; an alternative electric field controller; an alternative electric field amplifier; and an alternative electric field transmitter, wherein the alternative electric field generator generates an alternative voltage of 100 to 300 kHz and 1 to 20 Vpp, the controller sets an on/off time and period of the electric field to generate a discontinuous fractional alternative voltage, and the alternative electric field amplifier amplifies a voltage transmitted from the controller up to 800 Vpp, and the transmitter transmits the amplified voltage through.

According to the present invention, the on time and the off time of electric field each are preferably 1 minute to 24 hours, more preferably, 12 hours, but are not limited thereto.

According to the present invention, when the on/off that are each performed once is one period, the on/off period of electric field is preferably 1 to 7 periods, more preferably, 3 periods, but is not limited thereto.

According to the present invention, the alternative electric field amplifier amplifies the voltage transmitted from the controller up to 800 Vpp, the amplified voltage is transmitted to a target to which the electric field needs to be applied through the transmitter.

According to the present invention, an output frequency of alternative electric field is preferably 100 to 300 kHz but is not limited thereto.

According to the present invention, a substantial voltage of alternative electric field applied to cells is preferably 0.9 to 1.5 v/cm but is not limited thereto.

According to the present invention, an on/off time ratio of alternative electric field is preferably 1:1 but is not limited thereto.

Another aspect of the present invention relates to a method for treating cancer using an apparatus for suppressing a division of cancer cells or inducing a apoptosis of cancer cells by a system for generating a discontinuous fractional alternative electric field including: an alternative electric field generator; an alternative electric field controller; an alternative electric field amplifier; and an alternative electric field transmitter, wherein the alternative electric field generator generates an alternative voltage of 100 to 300 kHz and 1 to 20 Vpp, the controller sets an on/off time and period of the electric field to generate a discontinuous fractional alternative voltage, and the alternative electric field amplifier amplifies a voltage transmitted from the controller up to 800 Vpp and the transmitter transmits the amplified voltage to cancer to which an electric field needs to be applied.

According to the present invention, the method preferably comprises: a) generating an alternative voltage of 100 to 300 kHz and 1 to 20 Vpp in the alternative electric field generator; b) setting an on/off time and period of the electric field thereby generating a discontinuous fractional alternative voltage; c) amplifying the discontinuous fractional alternative voltage generated in step b) up to 800 Vpp in the alternative electric field amplifier; and d) transmitting the generated voltage to a treatment site using an electric pad.

According to the present invention, the cancer treatment suppresses the division of cancer cells or induces the apoptosis of cancer cells.

According to the present invention, in the on/off period of electric field, time for which the electric field is applied once and time for which the electric field is not applied is set to be one period.

The present invention relates to a method for treating cancer using an optimized discontinuous electric field when the electric field is applied to cells every three periods by setting the on/off time of electric field to one period of 0 h/24 h, 3 h/21 h, 6 h/18 h, 12 h/12 h, 24 h/0 h, or the like.

According to the present invention, the on time and the off time of electric field each are preferably 1 minute to 24 hours, more preferably, 12 hours, but are not limited thereto.

According to the present invention, the on time and the off period of electric field each are preferably 1 to 7 periods, more preferably, 3 periods, but are not limited thereto.

According to the present invention, the output voltage of alternative electric field is preferably 1 to 800 V and the output frequency of alternative electric field is preferably 100 to 300 kHz, but the output voltage and the output frequency of alternative electric field are not limited thereto.

According to the present invention, the substantial voltage of alternative electric field applied to cells is preferably 0.9 to 1.5 v/cm but is not limited thereto.

According to the present invention, the on/off time ratio of alternative electric field is preferably 1:1 but is not limited thereto.

The on/off time ratio of the present invention represents a ratio of the time for which the electric field is applied and the time for which the electric field is not applied.

A cancer treating method using an electric field that is currently being researched does not have a system for a method capable of protecting normal tissues, and therefore may not know how much the normal tissues are damaged by an electric field until side effects appear. However, the method for treating cancer using a discontinuous fractional alternative electric field according to the present invention makes a cell division time of cancer cells sensitive to an electric field shorter than normal cells while reducing an electric field applied to normal tissues, the method for treating cancer using a discontinuous fractional alternative electric field may obtain a treatment effect like the method for using a continuous alternative electric field. This may reduce the effect of electric field on the normal tissues to protect the normal tissues, thereby maximizing the treatment effect. Therefore, a sufficient intensity of electric field may be transmitted even to tumors that may not be treated due to the side effects of normal tissues at the time of treating cancer using the discontinuous fractional alternative electric field, and thus the current problem of treating cancer using an electric field may be improved.

### EXAMPLES

Hereinafter, the present invention will be described in more detail with reference to Examples. These Examples are only for illustrating the present invention and therefore it is apparent to those skilled in the art that the scope of the present invention is not to be constructed as being limited to these Examples.

### Example 1: Method and Apparatus for Treating Cancer Using Discontinuous Fractional Alternative Electric Field

To develop a system for protecting normal tissues from an electric field by treating cancer cells using a discontinuous fractional alternative electric field, a system for lowering an effect of suppressing a cell division of normal tissues less sensitive to an electric field while maintaining an effect of suppressing a division of cancer cells more sensitive to the electric field by adjusting the amount of electric field applied for the same treatment period.

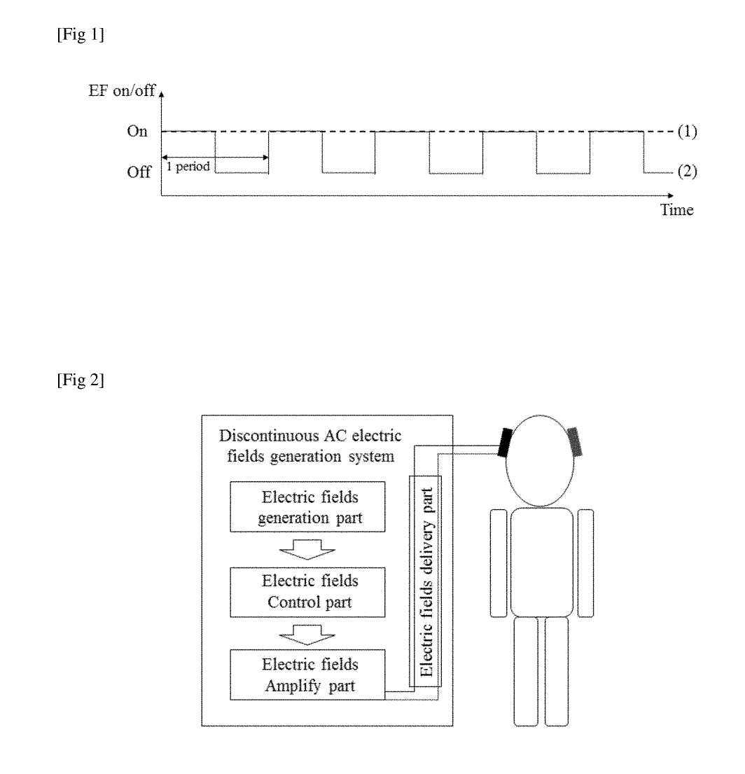

FIG. 1 is a graph illustrating a difference between the existing method for treating cancer using a continuous alternative electric field (FIG. 1, (1)) and a method for treating cancer using a discontinuous fractional alternative electric field (FIG. 1, (2)) proposed in the present invention. The existing method for treating cancer using a continuous alternative electric field applies an electric field over the treatment period, while the method for treating cancer using a discontinuous fractional alternative electric field performs a treatment by setting an on/off time of electric field, setting the on/off time of electric field as one period, and repeating the period over the treatment period. As a result, the method for treating cancer using a discontinuous fractional alternative electric field may perform a treatment with less electric field over the electric field used in the existing treatment method.

FIG. 2 is a schematic diagram of a system for generating and controlling a discontinuous fractional alternative electric field including an alternative electric field generator, an alternative electric field controller, an alternative electric field amplifier, and an alternative electric field transmitter.

### Example 2: Effect of Suppressing Division of Cancer Cells According to on and Off Time of Electric Field Per Day

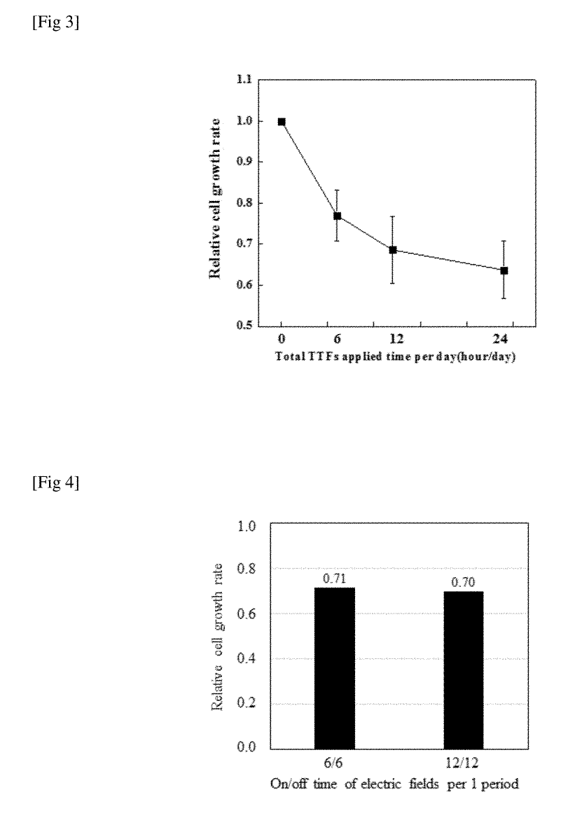

In the Example 2, it was confirmed how large the effect of suppressing a division of cancer cell (U373) is, in the case of using the discontinuous electric field over the treatment period of three days compared to the case of using the continuous electric field. In FIG. 3, a vertical axis represents a ratio of the number of cancer cells that suffers from the electric field treatment to the number of cancer cells (control group) that does not suffer from the electric field treatment and a horizontal axis represents a total time for which cancer cells are applied with an electric field per day and represents 0 hour/day, 6 hours/day, 12 hours/day, and 24 hours/day from the left. Total treatment time was 3 days, for all condition. Based on the analysis of the results, it was confirmed that a difference between cell growth rates in 0 hours/day and 6 hours/day is about 23% but a difference between cell growth rates in 12 hours/day and 24 hours/day that is twice as large as 0 hours/day and 6 hours/day in the difference in time for which the electric field is applied is rather reduced and thus is only 5%.

That is, it was confirmed that as the time for which the electric field is applied is long, the treatment efficiency against the treatment time is gradually reduced.

### Example 3: Effect of on/Off Time of Discontinuous Fractional Alternative Electric Field

In the Example 3, when a relative on/off time ratio of discontinuous fractional alternative electric field is the same as 1:1 but an absolute on/off time of discontinuous fractional alternative electric field is different, an effect of suppressing a division of cancer cells was compared.

In FIG. 4, a vertical axis represents a ratio of the number of cancer cells that suffers from the electric field treatment to the number of cancer cells (control group) that does not suffer from the electric field treatment and a horizontal axis represents results of treatment for 72 hours in total by making the on/off time ratio same as 1:1 but making the absolute time different as 12 hours/12 hours and 6 hours/6 hours. As a result, if the cell growth rate when the electric field is not applied is set to be 1, it was confirmed that the cell growth rate at 6/6 hours is 0.71±0.03 and the cell growth rate at 12/12 hours is 0.70±0.02.

That is, if the on/off time ratio of electric field is set to be 1:1, it was confirmed that the same electric field treatment effect is obtained even if the absolute on/off time is different.

### Example 4: Comparison of Discontinuous Electric Field Treatment Effect on Normal Cells and Cancer Cells

In the Example 4, the effects of the discontinuous electric field treatment on normal cells and cancer cells were compared and the conditions to show the same effect as the continuous electric field treatment effect on cancer cells but minimize the effect on normal cells were established.

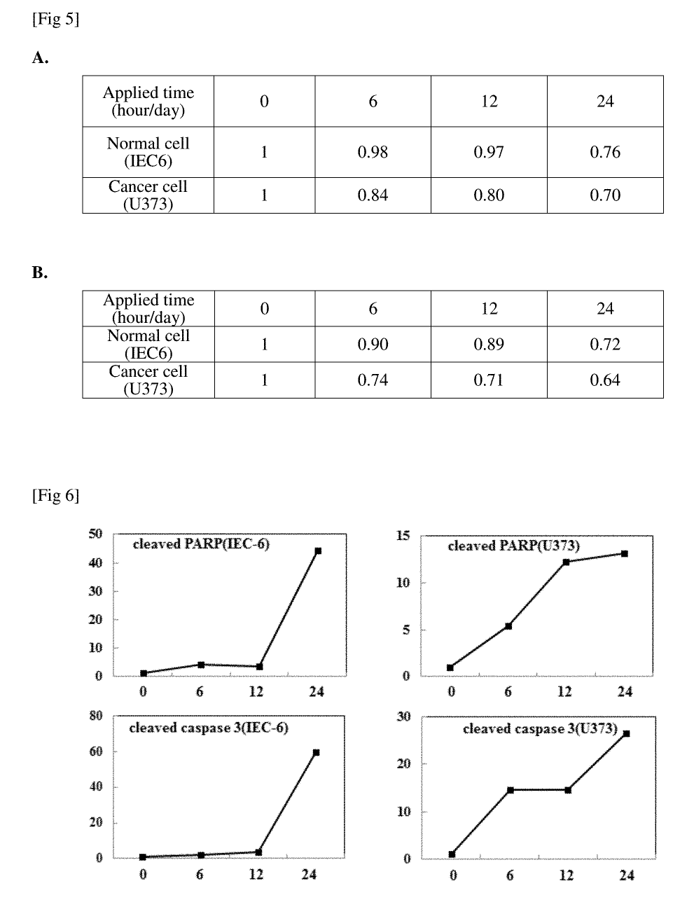

After an electric field of 0.9 v/cm and 1.2 v/cm is repeatedly applied to a normal cell (IEC6) and a cancer cell (U373) over three days by 0, 6, 12, and 24 hours per day, a cell growth rate was confirmed. (In FIG. 5, A represents a cell growth rate according to a time division condition when the electric field is 0.9 v/cm and B represents a cell growth rate according to a time division condition when the electric field is 1.2 v/cm) As the result, as illustrated in FIG. 5, it was confirmed that normal cells are less affected than cancer cells in all the experiment cases. Analyzing in more detail, it was confirmed that as the applied time is increased until 12 hours/day, the difference in growth rate between cancer cells and normal cells is gradually increased but is sharply reduced at 24 hours/day. Reviewing a result table of applying an electric field of 0.9 v/cm, in the case of 12 hours/day, the difference in growth rate between cancer cells and normal cells is 17% but in the case of 24 hours/day, is greatly reduced as 6%.

That is, because compared with the discontinuous electric field treatment, the continuous electric field treatment suddenly increases the damage to normal cells, the continuous electric field treatment needs to be avoided. To perform the optimized electric field treatment, it is necessary to find out a treatment condition to obtain the same effect as the continuous electric field treatment but minimize the damage to normal cells. As the confirmation result in the present experiment, as shown in two boxes of tables of FIG. 5, it was confirmed that a difference between a growth rate of cancer cells when an electric field of 0.9 v/cm is continuously applied and a growth rate of cancer cells when an electric field of 1.2 v/cm is applied only for 12 hours a day is little as only about 1% or less but a growth rate of normal cells when an electric field of 1.2 v/cm is applied only for 12 hours a day is 13% much higher than that of normal cells when an electric field of 0.9 v/cm is continuously applied. Therefore, instead of using the method for treating cancer using a continuous electric field, it is necessary to find out an optimized discontinuous electric field treatment condition capable of obtaining the same cancer treating effect but reducing damage to normal cells by increasing a level of electric field and discontinuously applying an electric field. If the condition is found, it is possible to greatly reduce side effects due to an electric field.

### Example 5: Confirmation of Effect of Discontinuous Fractional Alternative Electric Field Through Biological Factors

In the Example 5, when an electric field is applied to cancer cells and normal cells for 0, 6, 12, and 24 hours a day, the effects were confirmed through the biological factors.

FIG. 6 is a graph illustrating the amount of biological factors PARP and caspase-3 representing apoptosis based on a western blot. A vertical axis represents a relative amount of biological factor and a horizontal axis represents 0 hour/day, 6 hours/day, 12 hours/day, and 24 hours/day from the left as a set on/off time of electric field. It was shown that a cancer cell (U373) has a tendency that as time for which an electric field is applied a day is increased, the amount of biological factors is gradually increased but a normal cells (IEC6) has a tendency that the amount of biological factors is suddenly increased on a graph when an electric field is continuously applied.

That is, it was confirmed that normal cells are greatly damaged when an electric field is continuously applied, as compared with when an electric field is discontinuously applied.

According to the exemplary embodiments of the present invention, the discontinuous fractional alternative electric field shows the same effect as applying the continuous electric field to the cancer cells while reducing the effect on the normal cells, and as a result the method for treating cancer using an optimized alternative electric field may be constructed by the treatment using the discontinuous fractional alternative electric field.

Hereinabove, the specific portion of the present invention is specifically described but it is apparent to those skilled in the art that the detailed description is only a preferable embodiment and does not limit the scope of the present invention. Therefore, the substantial scope of the present invention is defined by the appending claims and equivalents thereto.

1. An apparatus for suppressing a division of cancer cells or inducing a apoptosis of cancer cells by a system for generating a discontinuous fractional alternative electric field including: an alternative electric field generator; an alternative electric field controller; an alternative electric field amplifier; and an alternative electric field transmitter, wherein the alternative electric field generator generates an alternative voltage of 100 to 300 kHz and 1 to 20 Vpp, the controller sets an on/off time and period of the electric field to generate a discontinuous fractional alternative voltage, and the alternative electric field amplifier amplifies a voltage transmitted from the controller up to 800 Vpp, and the transmitter transmits the amplified voltage through.

2. The apparatus of claim 1, the on/off time of electric field each are 1 minute to 24 hours.

3. The apparatus of claim 1, the on/off time of electric field each are 12 hours.

4. The apparatus of claim 1, the on/off period of electric field is 1 to 7 periods.

5. The apparatus of claim 1, the on/off period of electric field is 3 periods.

6. The apparatus of claim 1, the output voltage of alternative electric field is preferably 1 to 800 V.

7. The apparatus of claim 1, the output frequency of alternative electric field is preferably 100 to 300 kHz.

8. The apparatus of claim 1, the substantial voltage of alternative electric field applied to cells is 0.9 to 1.5 v/cm.

9. The apparatus of claim 1, the on/off time ratio of alternative electric field is 1:1.

10. A method for treating cancer using the apparatus of claim 1, the method comprises:
a) generating an alternative voltage of 100 to 300 kHz and 1 to 20 Vpp in the alternative electric field generator;
b) setting an on/off time and period of the electric field thereby generating a discontinuous fractional alternative voltage;
c) amplifying the discontinuous fractional alternative voltage generated in step b) up to 800 Vpp in the alternative electric field amplifier; and
d) transmitting the generated voltage to a treatment site using an electric pad.

11. The method of claim 10, the cancer treatment suppresses the division of cancer cells or induces the apoptosis of cancer cells.

12. The method of claim 10, the on/off time of electric field each are 1 minute to 24 hours.

13. The method of claim 10, the on/off time of electric field each are 12 hours.

14. The method of claim 10, the on/off period of electric field is 1 to 7 periods.

15. The method of claim 10, the on/off period of electric field is 3 periods.

16. The method of claim 10, the output voltage of alternative electric field is preferably 1 to 800 V.

17. The method of claim 10, the output frequency of alternative electric field is preferably 100 to 300 kHz.

18. The method of claim 10, the substantial voltage of alternative electric field applied to cells is 0.9 to 1.5 v/cm.

19. The method of claim 10, the on/off time ratio of alternative electric field is 1:1.
